(19) United States
(12) Patent Application Publication
(10) Pub. No.: US 2013/0035684 A1
(43) Pub. Date: Feb. 7, 2013

(54) Laser Treatment of Tissues at Wavelengths above 1330 nm

(71) Applicant: Wolfgang Neuberger, Dubai (AE)

(72) Inventor: Wolfgang Neuberger, Dubai (AE)

(73) Assignee: CeramOptec Industries Inc.

(21) Appl. No.: 13/197,975

(22) Filed: Aug. 4, 2011

(51) Int. Cl. A61B 18/22

(52) U.S. Cl. 606/41

(57) ABSTRACT

A medical laser system is disclosed for ablation/resection/coagulation of unwanted tissue, including parenchymal lung resection to facilitate/accelerate growth/wound healing. System comprises laser energy sources and conveying means, optical fiber. A diode laser source emits above 1330 nm, at least 50 Watts, through an optical fiber onto target tissue. Wavelength, 1340 nm is preferred. Wavelength ranges 1330-1390 nm and 1450-1550 nm, are also useful. Additionally a wavelength, between 800-1100 nm, can be used. Wavelength differences maximize beam quality. Two radiation sources emit simultaneously from fiber's distal end. One emits at ˜1320 nm and the other emits at ˜1340 nm or ˜1360 nm with maximum total output power of 60 W or larger. Preferably, the ratio of the power levels is fixed at 1:1.5 and output power is the sum of individual lasers. High beam quality and power density system combines emissions from a diode laser and a fiber laser.

### BACKGROUND OF THE INVENTION

1. Field of the Invention

The present invention relates to laser medical treatments. More particularly, it relates to fiber coupled medical laser systems and methods used for surgical treatment of medical conditions employing lasers operating at or above 1330 nm.

2. State of the Art

Metastatic tumors in the lungs are cancers that develop at other places in the body (or other parts of the lungs) and spread through the bloodstream or lymphatic system to the lungs.

The lungs are the second most frequent site of metastases and often the only location of metastatic disease. Pulmonary metastases are common and most frequently occur with tumors that have rich systemic venous drainage. Metastatic disease to the lung is common because the entire output of the right heart and the lymphatic system flow through the pulmonary vascular system. Since the entire cardiac output flows through the lungs, the risk of hematogenous metastases is very high. Studies have demonstrated that pulmonary metastases are present in 20-54% of all patients who die of cancer.

Excluding primary lung cancers, which metastasize to either lung, tumors involving the lung parenchyma are breast cancer, gastrointestinal tumors, kidney cancer, melanoma, sarcomas, lymphomas and leukemias, germ cell tumors, and rarely ovarian cancer.

The initial event occurs at the primary tumor site. Fragments of tumor are dislodged after venous invasion, and they are carried as tumor emboli to the lungs via the systemic circulation. The majority of these fragments lodge in the small pulmonary arteries or arterioles, where they may proliferate and extend into the lung parenchyma and ultimately form nodules. These nodules are most commonly located either subpleurally or in the lung bases rather than in the upper lung, locations that reflect the pulmonary arterial circulation.

Usually cancer will be present even in places not seen by CT scans. In these circumstances, removing the visible tumors with surgery is usually not beneficial, although it may still be considered. Chemotherapy is usually the treatment of choice. But when the primary tumor has been removed and the cancer has spread to only limited areas of the lung, the lung tumors can be removed with surgery. Metastases are resected to increase the disease-free survival.

Surgical resection of pulmonary metastases is now common and is a routine part of thoracic surgical practice. Lung resection can also be done to address congenital abnormalities and other problems related to the lungs, such as abscesses in the lung. Resection of indeterminate pulmonary lesions in patients with a history of malignancy is indicated, as the presence of metastases will provide prognostic information and often dictate further therapy. Pulmonary metastasectomy also improves survival in select patients with favorable tumor histologies.

Techniques chosen for surgical resection of diseased lung tissue seek to minimize undesired effects such as bleeding and air leak due to incision. Ultrasonic devices are unsatisfactory for this purpose. Stapling devices are used to try to overcome bleeding and air fistulae by pressing the lung tissue between two rows of clamps. However the shape of staple wedge makes technique lack precision.

Laser systems can be beneficial and effective medical instruments. They allow specific treatment to be administered and are frequently preferred by those skilled in the art. Thus, there is an increasing number of medical applications involving the use of laser devices.

Surgical applications involve ablation of unwanted tissue, such as cancerous tissue. Such tissue ablation causes excessive bleeding, and therefore must simultaneously be accompanied by means to achieve effective homeostasis. Two key features of laser radiation to achieve optimum results are power and wavelength.

Laser treatments have been improved due to new diode laser systems. Diode laser systems are greatly advantageous in comparison with other existing laser source technologies such as CO₂, holmium:YAG, pulsed-dye, Nd:YAG or KTP laser sources, in that they provide higher output, at reduced dimensions and weight. They also have simpler and smaller air cooling systems. Moreover, being integrated with optical fibers, they have a high reliability and do not need alignment. In summary, they provide great efficiency and robustness. Consequently, laser diode devices are commonly used for treatment of metastasic highly vascularized tissue such as lung or liver tissue.

The lung's parenchyma has a very high percentage of water as well as high vessel density. Resection of lung tissue thus requires a laser with a combination of great cutting and coagulation capability. From the absorption curves of different wavelengths in water, it can be observed that at absorption peaks, applied energy is immediately transformed into tissue vaporization while little or no significant coagulation occurs. Therefore the Er:YAG and CO₂ lasers are unsuitable for use on the lung parenchyma. Holmium laser, is highly absorbent at 2100 nm and thus coagulation capacity may be limited.

In US Patent Application US20070219601A1 by Neuberger, a device and method are disclosed, which achieve tissue ablation as well as tissue coagulation simultaneously during the treatment of BPH by utilizing at least two wavelengths of light. Tissue ablation is affected by having one wavelength that is highly absorbed in the prostatic tissue while another less highly absorbed wavelength coagulates surrounding tissues while maintaining minimal thermal damage to surrounding tissue. Highly absorbed wavelength is about 1460 nm and the less absorbed by water but with significant hemoglobin absorption is about 980 nm.

When treating soft tissue including pulmonary tissue using a wavelength between 1100 nm and 1400 nm, an absorption of the laser irradiation in aqueous tissue, sets in, which allows to efficiently separate or cut the tissue while at the same time generating an expanded coagulation on severely blood-supplied tissue. US Published Patent Application No. 2008/0009844A1 by Rolle et al. refers to a device for laser surgery comprising a laser, and means for coupling light of the laser into an optical fiber, wherein the laser is a solid-state laser, which may emit laser light which at maximum intensity has a wavelength in the range of 1100 and 1400 nm, and the laser has an output power of at least 25 W. More particularly, patent claims a wavelength range where maximum intensity is above 1150, 1175, 1200, 1275, 1300, 1308 or 1310 nm.

Laser systems that emit a wavelength of 1318 or 1320 nm have been known since quite some time ago, because such wavelengths are available from Nd:YAG or similar materials. In several publications such as Laser Applications in Lung Parenchyma Surgery, Med. Laser Appl. 18: 271-280 (2003), Rolle discloses a laser emitting the 1318 nm wavelength exclusively up to a power of 40 W. This is achieved with an Nd:YAG nm wavelength laser system that allows removal of a significantly higher number of lung nodules in comparison to conventional techniques.

Disclosed devices emit determined maximum power levels at only 1 wavelength. Thus, they may lack effectiveness and versatility as treated tissues require radiation that causes both ablation and coagulation effects. Lack of versatility also means limitations to treating other types of soft tissue such as for example liver tissue or kidney tissue. Finally, undesired effects on tissue are still reported with mentioned currently available devices. Thus physicians seek better beam quality, i.e. a smaller focus point, longer focal length of handpiece, higher and more consistent power density on tissue or fiber tips.

There is therefore a need for a laser surgical system that improves on the state of the art by allowing more precise and effective resection and coagulation of unwanted soft tissue such as metastases cancerous tissue. The present invention addresses this need.

### OBJECTIVES AND BRIEF SUMMARY OF THE INVENTION

It is an objective of the present invention to provide a device and method for improved surgical procedures such as lung metastasis treatments.

It is another objective of the present invention to treat lung metastasis effectively by one or more localized, directed energy sources and conveying means.

It is also an objective of the present invention to adequately combine light of different wavelengths to achieve optimal resection and coagulation effects on soft tissue.

It is yet another objective of the present invention to provide a device and method of lung metastasis treatment by providing precise and effective resection and coagulation of soft cancerous tissue with minimum side effects.

Briefly stated, a fiber coupled medical laser system is disclosed for ablation/resection and coagulation of unwanted tissue by using one or more local energy sources. When applied to the lung treatment is based on parenchymal lung resection to facilitate new tissue growth and accelerate the wound healing process. System preferably comprises one or more laser energy sources and conveying means such as an optical fiber with a proper emitting tip configuration. An imaging system may be incorporated into system or a standalone imaging device such as an endoscope or laparoscope may be employed. In a preferred embodiment, a diode laser source emits at a wavelength of over 1330 nm and laser energy at 50 Watts or more, which is conveyed through an optical fiber and applied on target tissue, such as lung tissue. A preferred wavelength is about 1340 nm. In other embodiments, system emits over wavelength ranges such as 1330 to 1390 nm and 1450 to 1550 nm. In another embodiment, system includes an additional wavelength in the range of 800 to 1100 nm. The difference in wavelength of the different diodes is utilized to maximize the beam quality through suitable combination means. In another embodiment, two combined radiation sources emit simultaneously from a common fiber's distal end. One wavelength emits at about 1320 nm and a second wavelength emits at about 1340 nm or about 1360 nm at a maximum total output power of 60 W or larger. Preferably, the ratio of the power levels is fixed at 1:1.5 and the output power is the sum of each individual power source. In another embodiment, medical laser system provides high beam quality and power density by combining emission from a diode laser source with emission from a fiber laser source.

The above and other objects, features and advantages of the present invention will become apparent from the following description read in conjunction with the accompanying drawings (in which like reference numbers in different drawings designate the same elements).

### BRIEF DESCRIPTION OF FIGURES

FIG. 1 depicts the main components of a preferred embodiment of present invention. FIGS. 2a and 2b show laser radiation absorption curves for water and blood.

FIG. 2c depicts penetration of laser light between 900 and 1380 nm for different tissues.

FIG. 3 depicts main components of another preferred embodiment of present invention with two diode laser sources.

FIG. 4 depicts main components of another preferred embodiment of present invention comprising a fiber laser source and a diode laser.

### DETAILED DESCRIPTION OF PREFERRED EMBODIMENTS

Present invention describes a medical laser device for treatment of cancerous tissue such as lung metastases based on lobe-sparing resection of such tissue to facilitate new tissue growth and accelerate the wound healing process.

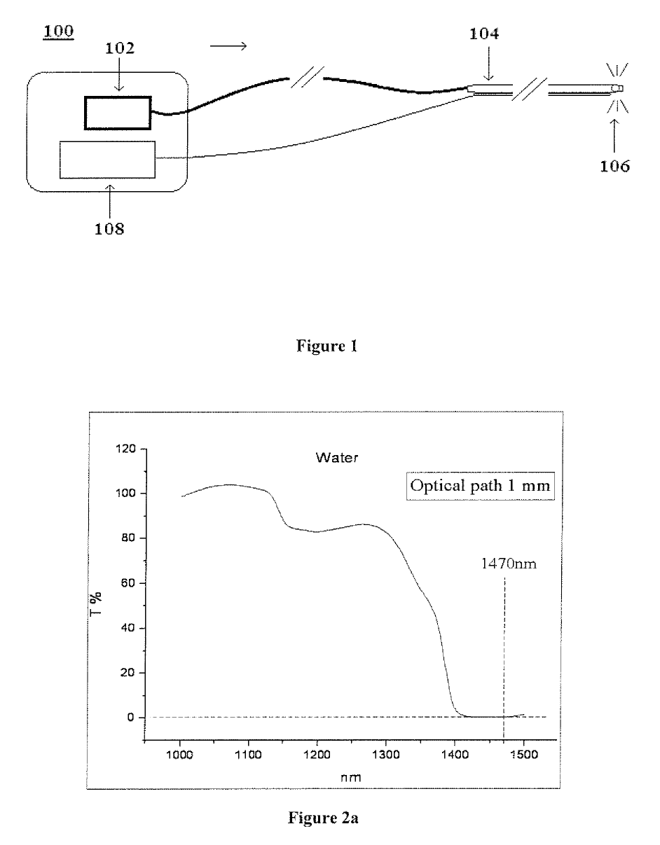

In a preferred embodiment of present invention, depicted in FIG. 1, laser based system 100 comprising laser source 102 emits at a suitable wavelength through fiber optics device 104 radiating from its distal end 106 in a pattern according to chosen fiber tip (radial, twister, forward, etc). Fiber optics device 104 is applied on soft tissue utilizing suitable tools for imaging, placement and insertion, such as an endoscope or laparoscope to investigate and adjust necessary laser energy deposition. Imaging system 108 may be incorporated into fiber optics device 104 or a standalone imaging device may be employed and may include but is not limited to ultrasound, camera vision, magnetic resonance tomography, or computed tomography sets. This allows for an on-line monitoring of the resection effect and for applying proper energy dosage into target tissue to prevent undesired consequences of under or overtreatment.

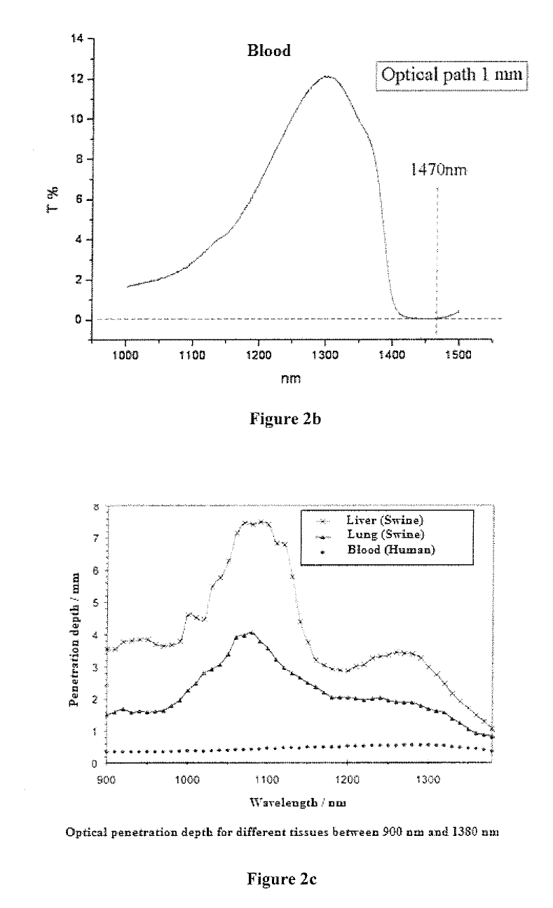

FIGS. 2a and 2b show results obtained in a search for a preferred wavelength to use in present invention, where clinical trials were carried out and transmission spectra in water (FIG. 2a) and in blood (FIG. 2b) were obtained for a wavelength range between 1000 nm and 1500 nm in an optical path of 1 mm. FIG. 2c depicts penetration of laser light between 900 and 1380 nm for different tissues, as published by Knappe et. al in Radiologe 2004 44:677-683 “Lasertherapie in der Lunge: Biophysikalischer Hintergrund”. Curve formed by triangle-shaped dots 314 represents results obtained in trials on a pig's lung. Authors state that in-vivo lung tissue would have larger penetration depths as air fills the lung and the light can travel through the air-filled areas without being absorbed. From FIGS. 2a-2c, it can be observed that at 1340 nm or at 1360 nm, vaporization of tissue would be achieved using a lower output power than for example at 1320 nm. This is because 1320 nm-radiation is absorbed less efficiently than 1340 nm or 1360 nm. Also, radiation at those longer wavelengths causes a larger coagulation zone, so coagulation of blood with 1340 nm or 1360 nm are at least 20% more effective than with 1320 nm. Additionally, at 1450 nm or above, almost total absorption in blood and water takes place. Penetration depth in lung tissue at these wavelengths is near 1 mm, which is enough to reach and attack cancerous lung tissue.

Thus, in a preferred embodiment, treatment system emits the maximum of its output power at a wavelength above 1330 nm. Preferably, system can emit at a wavelength ranging from 1340 to 1390 nm or 1450 to 1550 nm. Maximum output power is preferably above 50 watts.

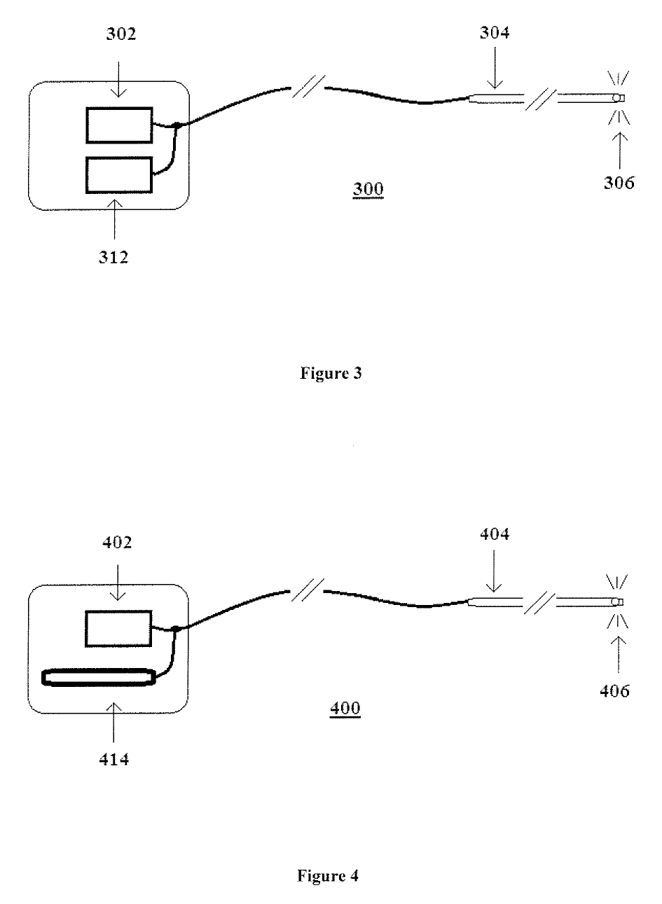

FIG. 3 depicts another preferred embodiment of present invention. System includes laser based system 300 comprising at least two laser sources combined into one fiber optic device 304. First laser source 302 emits at a wavelength of about 1320 nm with a maximum output power (P₁ₘₐₓ) of preferably 40 Watts or less. Second laser source 312 emits at a wavelength between about 1340 and 1400 nm at a maximum output power (P₂ₘₐₓ) greater than first wavelength's maximum output power, that is: P₂ₘₐₓ>P₁ₘₐₓ. All wavelengths are transmitted through same waveguide 304 and are focused on the same spot. Combined radiation sources emit from common distal end 306 in a pattern according to chosen fiber tip (radial, twister, forward, etc). In preferred embodiments, second wavelength emits at about 1340 nm or about 1360 nm at a maximum output power of 60 W or larger. Preferably, the ratio of the power levels P₁: P₂ is fixed and said ratio is preferably P₁: P₂=1:1.5. Radiation at both wavelengths is emitted simultaneously. Thus, the total output power level Pₜₒₜₐₗ is the sum of each individual source power so: Pₜₒₜₐₗ=P₁+P₂, and the total maximum power level P_{total max} becomes: P_{total max}=P₁ₘₐₓ+P₂ₘₐₓ. In other preferred embodiments, different combinations of emitting wavelengths from sources 302 and 312 are applied, including but not limited to 1350 nm+1470 nm, 1350 nm+1550 nm, 1320 nm+1350 nm, and 1350 nm+1900 nm.

In another preferred embodiment, system comprises additional radiation sources operating in the range of 800 to 1100 nm. This way, a wavelength for coagulation can be suitably mixed in to help in applications where bleeding can be a particular problem such as in lung metastases treatment. Preferably, optical fibers supplied from laser sources are bundled together so as to emit radiation at a common tip through a proper handpiece.

In another preferred embodiment, system comprises a detachable handpiece and different specific handpieces that can be coupled to fiber or fiber bundle according to fiber tip emission characteristics. This way, surgery device can be alternatively modified to emit radiation in a forward direction, off-axis, or radially at 360 degrees to name some examples. The present system is, thus, useful for its versatility in adapting to different anatomic and geometric targets.

Tissue effects and treatment results are positively enhanced by higher wavelengths used with present invention. Furthermore, treatment tissues would benefit from better beam quality (smaller focus points, longer focal length of handpiece, higher and more consistent power density on tissue or fiber tips) achievable by combining and overlapping wavelengths. Thus, wavelengths for coagulation can be suitably mixed in to help in certain applications where bleeding can be a particular problem.

Fiber laser emission sources, also called laser-active fibers, generate laser radiation if they are optically pumped, for example by another laser. They consist of a core/clad structure wherein the core is doped with rare earth ions such as neodymium, ytterbium and erbium. Thus, optical pump radiation of appropriate wavelength and power is launched into a laser-active fiber, and the rare earth ions will absorb the pump radiation and will start to emit laser radiation themselves. One major advantage of a fiber laser is its possibility to generate single-mode laser radiation which renders excellent beam quality at high power levels. Another advantage is the fiber laser's capability of generating laser radiation at wavelengths that cannot be targeted by laser diodes directly.

FIG. 4 depicts another preferred embodiment of present invention. System includes laser based system 400 comprising diode laser source 402 in combination with fiber laser emission source 414. Each source emits at a suitable wavelength through fiber optics device 404 radiating from its distal end 406 in a pattern according to chosen fiber tip (radial, twister, bare, etc).

In a preferred embodiment, combination of a diode laser wavelength emission with a fiber laser emission is achieved by discrete beam combining. In another preferred embodiment, combination of a diode laser wavelength emission with a fiber laser emission is achieved by a system where laser emission at one wavelength is the residual pump radiation that was not absorbed by the laser-active fiber where the second wavelength is emitted from the fiber laser itself. The pump radiation can be emitted from the pump core or the laser-active core itself. In another preferred embodiment, combination of a diode laser wavelength emission with a fiber laser emission is achieved by a system where pump radiation and laser radiation at an additional wavelength are launched into the laser-active fiber. The pump radiation is used to stimulate the fiber laser and the fiber laser radiation and the laser radiation at the additional wavelength are extracted from the fiber laser.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A fiber coupled medical laser system for irradiating and treating soft human tissue comprising:
at least one radiation source capable of emitting at a wavelength above 1330 nm:
at least one fiber optic delivery means, having a proximal end and a distal end;
wherein said radiation source is capable of producing radiation at a preselected wavelength and a preselected power.

2. The system according to claim 1, further comprising an interchangeable handpiece permitting the use of different emitting tip configurations.

3. The system according to claim 1, further comprising an imaging system.

4. The system according to claim 1, wherein said at least one radiation source is capable of emitting the maximum of its output power at a wavelength above 1330 nm.

5. The system according to claim 4, wherein said maximum output power is above 50 watts.

6. The system according to claim 4, wherein said wavelength is between about 1330 and about 1390 nm.

7. The system according to claim 4, wherein said system emission is between about 1450 and about 1550 nm.

8. The system according to claim 4, further comprising at least one additional source emitting at a wavelength in the range of 800 to 1100 nm.

9. The system according to claim 4, wherein a difference in wavelength of the different diode lasers is utilized to maximize the beam quality through a suitable combining means.

10. The system according to claim 1, wherein said at least one radiation source comprises two radiation sources emitting simultaneously at power P₁ and P₂, respectively and at a fixed power ratio r=P₁/P₂ and where the total power emitted Pₜₒₜₐₗ is equal to the sum of P₁ and P₂ and where the total maximum power emitted Pₜₒₜₐₗₘₐₓ is equal to the sum of the maximum power emitted from each radiation source P₁ₘₐₓ and P₂ₘₐₓ.

11. The system according to claim 10, wherein said ratio r is preferably equal to 1.5.

12. The system according to claim 1, comprising two radiation sources emitting simultaneously at a combination of wavelengths selected from the group of 1350 nm+1470 nm, 1350 nm+1550 nm, 1320 nm+1350 nm, and 1350 nm+1900 nm.

13. The system according to claim 1, wherein said at least one radiation source comprises two radiation sources, one radiation source emitting at about 1320 and an additional source emitting in the range of about 1340-1360 nm.

14. The system according to claim 1, wherein said at least one radiation source comprises a diode laser and a fiber laser, wherein said laser emissions are combined at system's distal end.

15. A method of using the medical treatment system according to claim 1, wherein said soft tissue is highly vascularized tissue.

16. The method of using the medical treatment system according to claim 15, wherein said highly vascularized tissue comprises lung, kidney or liver tissue.
